(19) United States
(12) Patent Application Publication
(10) Pub. No.: US 2005/0208108 A1
(43) Pub. Date: Sep. 22, 2005

(54) Thermoplastic films and methods for making

(76) Inventors: Leonard C. Jannusch, White Bear Lake, MN (US); William L. Bunnelle, Andover, MN (US); Robert M. Hume III, Woodbury, MN (US)

Correspondence Address: Merchant & Gould P.C., P.O. Box 2903, Minneapolis, MN, 55402-0903, US

(21) Appl. No.: 10/805,172

(22) Filed: Mar. 19, 2004

(51) Int. Cl. A61K 9/68; A61K 9/70

(52) U.S. Cl. 424/443; 424/440

(57) ABSTRACT

A thermoplastic film that is configured to disperse in contact with a body fluid of a live human or other animal. The film includes a thermoplastic film former and a fatty alcohol. The film is produced by extruding or otherwise coating a coatable composition. For some applications, the film is edible, and disperses upon contacting the film to the eater's tongue. The edible film can include a breath freshening agent, a medicament or other substance. The film can also be used topically or in a mucous membrane to deliver a medicament.

### FIELD OF DISCLOSURE

The present disclosure relates to dispersible polymeric films, including edible films, and methods of making these films. More specifically, this disclosure relates to dispersible thermoplastic films, including edible films.

### BACKGROUND OF THE DISCLOSURE

There are several dispersible film products available that provide various desired features such as breath freshening and other oral hygiene properties when placed in the mouth of the user. The term dispersible is used to mean that the film is configured to dissolve or break apart into small pieces. The film product is typically small, configured to fit on a person's tongue. The film product is sufficiently rigid to handle and delays softening until placed in the mouth. Upon exposure to saliva, the bulk of the film product disperses, releasing the functional ingredients. The term disperse is used to mean dissolve or break apart into small pieces.

The film compositions that are used to make these film products are often aqueous based, made from a polymeric material that is made into a solution with various ingredients such as gums, thickeners, flavorants, and the like, dissolved in water or a water and ethanol solvent. This film composition is typically cast onto a liner and then dried. Due to the high percentage of volatile substance in the film composition, the coated film cannot be quickly dried of water and any solvent. The drying process consumes considerable time and energy.

What is desired, therefore, is an acceptable film product that is more conducive to quick manufacturing.

### SUMMARY OF THE DISCLOSURE

The present invention is directed to a thermoplastic film that is dispersible when in contact with a body fluid. The film is biologically compatible, so that it is capable of being assimilated into the body. For some applications, the film is edible, and disperses upon contacting the film to the eater's tongue. The edible film can include a breath freshening agent or, medicament or a nutrient. For other applications, such as topical applications or in a mucous membrane, the film can include a medicament.

The coatable composition of the present invention includes a thermoplastic film former and a fatty alcohol. The moisture content in the film is generally less than 10 wt-%. Other additives, such as fillers and flavorants can be added.

The coatable composition is extruded from a molten state or otherwise coated to form a coated film and then cooled. The coatable composition is a thermoplastic composition. Upon cooling, the composition quickly forms a cooled coated film that is non-tacky to the touch. Usually, the coated film is non-tacky within two to ten seconds of coating. The coated film is then cut into the film product. The term film as used herein refers to a thin flexible sheet of material and encompasses the coated film, the cooled coated film, and the film product. The term coated film refers to the film after it has been coated onto a substrate, while it remains with the substrate.

In one embodiment, the invention is a film resulting from a coating of a composition comprising hydroxypropyl cellulose and at least 10 wt-% fatty alcohol component by total film weight. The film is thermoplastic and is configured to disperse in contact with a body fluid of a live human or other animal in 5 minutes or less. The film has a thickness of 1 mm or less and a perimeter area of 15 square cm or less.

In another embodiment, the invention is a film resulting from a coating of a composition that includes:
(a) 5 to 50 wt-% hydroxypropyl cellulose by total film weight,
(b) 5 to 60 wt-% fatty alcohol component by total film weight, and
(c) 15 to 80 wt-% dextrose by total film weight.
   The film is thermoplastic, has a thickness of 1 mm or less, and has a perimeter area of 15 square cm or less.

In another embodiment, a film resulting from a coating of a composition includes:
(a) 10 to 25 wt-% hydroxypropyl cellulose by total film weight,
(b) 20 to 50 wt-% stearyl alcohol by total film weight,
(c) 40 to 60 wt-% dextrose by total film weight, and
(d) 1 to 5 wt-% artificial sweetener by total film weight.

The film has a thickness of 0.03 to 0.1 mm and is thermoplastic. The film is orally ingestible and is configured to disperse in a mouth of a consumer within 30 seconds or less.

A method of making a film includes the steps of providing a water-soluble film former, mixing the film former in a fatty alcohol component to form a mixture, and extruding the mixture to form a coated film, where the mixture is at a temperature of at least 65 degrees C. when extruded. In one embodiment of the film, it is made using these method steps. Preferably, when the extruded film cools to 30 degrees C. or lower, the extruded film is non-tacky.

A method of making a film includes the steps of providing a water-soluble film former, mixing the film former with a substance to form a mixture having less than 5 wt-% water, and extruding the mixture to form a film, wherein the mixture is at a temperature of at least 65 degrees C. when extruded.

### BRIEF DESCRIPTION OF THE DRAWINGS

FIG. 1 is a schematic depiction of a container having a plurality of film product pieces therein;

FIG. 2 is a perspective view of a single film product; and

FIG. 3 is a schematic diagram of a coating apparatus suitable for extruding the coated film of the present invention.

### DETAILED DESCRIPTION OF THE PREFERRED EMBODIMENT

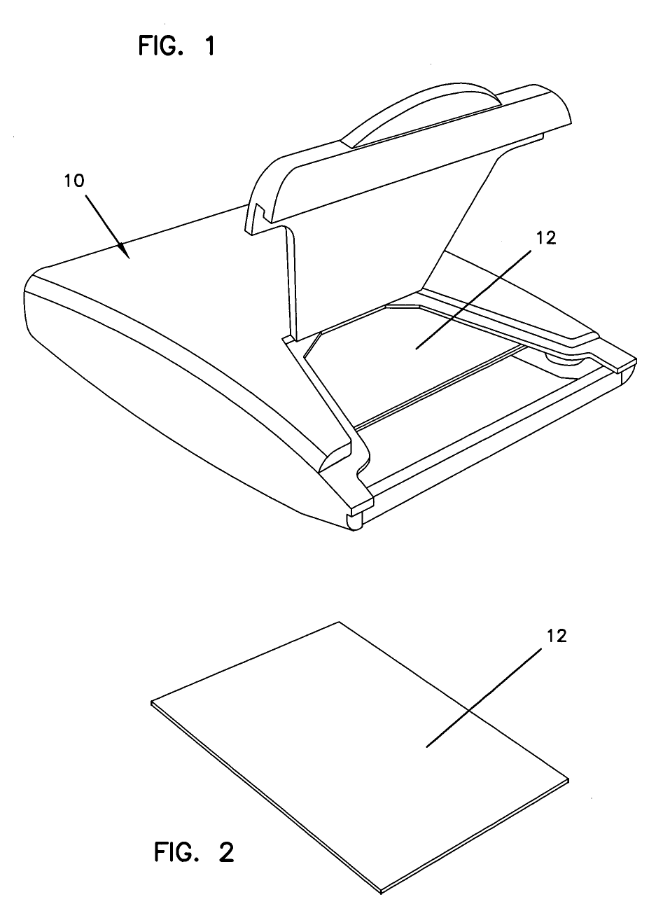

Referring to the figures, FIG. 1 illustrates a container 10 having a plurality of film product pieces 12 retained therein. A single film product 12 is illustrated in FIG. 2. In one embodiment, film product is designed to be orally ingested by a consumer. In other embodiment, film product is designed to disperse in a topical application on skin, or in contact with a mucous membrane, or in contact with a body fluid such as saliva or blood of a human or other animal.

Examples of conventional products similar to that illustrated in FIGS. 1 and 2 include “Listerine Cool Mint Pocket Paks”, which are breath freshening strips from Pfizer Consumer Healthcare, “Chloraseptic Sore Throat Relief Strips”, which are sore throat relief strips from Prestige Brands International, Inc., and “Altoids Strips”, which are breath freshening strips from Callard & Bowser-Suchard, Inc., and “Fresh Breath Strips”, which are also breath freshening strips from Walgreen Co.

For the configurations illustrated in FIGS. 1 and 2, film product 12 is rectangular and has height and width dimensions of 1-4 cm and is 0.03 to 0.1 mm thick, usually 0.05 to 0.08 mm thick. A typical film product 12 weighs approximately 0.3 grams.

The term film refers to a thin flexible sheet of material and is intended to encompass the coated film, the cooled coated film and the film product. The film has a thickness sufficient to be rigid enough so that film product pieces with a perimeter area of 2-15 square cm are self-supporting, so that they do not bend under their own weight when grasped at one part, typically, at least 0.01 mm thick. The film typically has a thickness of no more than 2 mm, more typically no more than 1 mm. A thickness of 0.03 to 0.5 mm is preferred, depending on the use of film 12. For edible applications, a thickness of 0.03 to 0.1 mm is preferred.

In one embodiment, the film product has a perimeter area of 15 square cm or less, or, more preferably, 10 square cm or less. The film product preferably has a perimeter area of 4-8 square cm for edible films. Many different sizes for the film can be used, depending on the application.

The film product of the present invention can have many different shapes including rectangular, circular, oval, triangular, animal shapes or other fanciful shapes. The film products are often rectangular with a length and width at least 0.5 cm, usually at least 1 cm. The length and width are usually no greater than 30 cm, generally no greater than 20 cm. For edible applications, the length and width are usually 1 cm to 4 cm. In one particular embodiment, film product is 2.25 cm by 3 cm. In another particular embodiment the film product is 2 cm by 3 cm.

The film product in general, is thermoplastic, with adequate film integrity and rigidity to withstand handling. By use of the term “thermoplastic”, what is intended is a polymer that softens and flows when exposed to a selected level of heat above room temperature and returns to its original rigid condition when cooled to a selected temperature, for example room temperature.

In addition, a thermoplastic material can be softened and solidified without undergoing an appreciable chemical change if the temperature of the material does not exceed some temperature, such as 275° F. (135° C.). For example, a thermoplastic material does not degrade or char when heated to a temperature of 135° C. or below.

Preferably, the film is solid at temperatures up to at least 100° F. (38° C.), more preferably it is solid up to at least 130° F. (54° C.). In one embodiment, the film begins to soften and flow at a temperature that is higher than 130° F. (54° C.). In a more preferred embodiment, the film begins to soften and flow at a temperature that is equal to or higher than 180° F. (82° C.), more preferably 200° F. (93° C.) or higher.

Preferably the film is configured to disperse upon exposure to water or a body fluid. In one embodiment, film pieces 12 are non-blocking so that they can be stacked on one another without sticking together at temperatures of up to 130° F. (54° C.). In other embodiments, it is desirable for the film pieces to have an adhesive quality at room temperature so that they can stick to the skin or other surface.

Those film products intended for oral ingestion preferably have a pleasant or desirable taste. The film former, viscosity modifier, and other ingredients apart from any sweeteners or flavorants present in the film product can have a neutral taste that is not detectable to a consumer. Additionally, it is desired that the film disperses within a few minutes, preferably within 30 seconds, when exposed to saliva.

Film product pieces 12 of the present invention are made from a thermoplastic coatable composition. In accordance with the present invention, the coatable composition is a liquid mixture at a temperature higher than typical room temperatures. The composition is extruded or otherwise coated to form a coated film, and cools to room temperature to form a cooled coated film. This film can be cut into film product. The coatable composition of the present invention includes a thermoplastic film former, a viscosity modifier as a solvent, and any ingredients for tailoring the film for its intended use.

No more than 10 wt-% of the coatable composition is water based on the total weight of the coatable composition. In one embodiment, the water content in the coatable composition is generally no more than 5 wt-%, and typically no more than 2 wt-% based on the total weight of the composition. The film has no more than 5 wt-% water, preferably no more than 2 wt-% water, and more preferably no more than 0.5 wt-% water based on the total weight of the coated film. In one embodiment there is no detectable level of water in the coated film.

The film product is not very sensitive to humidity changes, and is not likely to curl in response to changes in humidity. As a result, more materials are available for packaging the film pieces inexpensively.

In one embodiment, the film has dimensional stability and does not significantly expand in response to humidity changes. For example, in one embodiment, the film will expand or contract less than 30% and preferably less than 20% in any one dimension in response to a change in humidity. In another embodiment, the film will expand or contract less than 10% and preferably less than 5% in any one dimension in response to a change in humidity.

As stated above, the coatable composition is a thermoplastic composition. After forming the coatable composition, which is done in a liquid state to facilitate mixing of the ingredients, the composition can be immediately coated, or, the composition can be solidified for later coating. If storage of the composition for later coating is desired, the coatable composition can be solidified into blocks, bricks, pellets, or any other suitable form. The solid composition can be crushed, grated, or otherwise reduced in size to facilitate subsequent melting and coating.

Examples of Film Uses

In one example of an application of the film product of the present invention, the film product is orally consumed for freshening breath or for delivering a substance to the consumer. In this application context, a film piece that is 0.05 mm thick, 2.25 cm wide and 3 cm long will preferably disperse when in contact with saliva in a consumer's mouth in less than 5 minutes, or more preferably less than 2 minutes. Preferably, the film is configured to disperse in contact with saliva within a time period less than 1 minute, or more preferably 30 seconds or less.

In another application, the film product can be a component of a bandage or dressing that retains an active ingredient in powder form in place in the bandage. When the film product contacts the blood or other fluid of the wound, it disperses, releasing the active ingredient to the wound. Alternatively, the film product can be applied to a wound to deliver an active ingredient to the wound site. In this application context, a film piece that is 0.05 mm thick, 2.25 cm wide and 3 cm long will preferably disperse when in contact with the blood or other fluid of the wound in a time period less than 5 minutes, or more preferably less than 2 minutes. Preferably, the film product is configured to disperse in use in contact with the body fluid within a time period less than 1 minute, or more preferably 30 seconds or less.

In another application example, the film product can be applied to skin to deliver an active ingredient that is beneficial to the skin. In order to release the active ingredient, the user rubs the film product with a wet finger moistened with saliva or water, causing the film product to disperse. In this application context, a film piece that is 0.05 mm thick, 2.25 cm wide and 3 cm long will preferably disperse when moistened in a time period less than 5 minutes, or more preferably less than 2 minutes. Preferably, the film product is configured to disperse within a time period less than 1 minute, or more preferably 30 seconds or less. Many other applications for the film exist beyond those few discussed herein.

Film Former

The coatable composition of the present invention includes a polymeric film former that is water soluble or water dissolvable. Additionally, the film former is a thermoplastic material. For edible film products, the film former preferably meets the appropriate FDA regulations, and is tasteless and odorless.

Examples of suitable thermoplastic film formers for the present invention include hydroxypropyl cellulose, polyoxyethlene poly(2-ethyl-2-oxazoline) and similar polymers, polyvinylpyrrolidone, vinyl acetate, and mixtures thereof. Other film formers that are suitable include carboxymethyl cellulose and pullulan. Hydroxyl waxes can also be suitable as the film former in coatable compositions and in the coated film product of the present invention. Mixtures of these film formers can be used.

The film former is present in the coatable composition at a level of at least 2 wt-%, and typically at least 5 wt-% based on the total weight of the compositon. Usually, no more than 50 wt-% of the coatable composition is the film former, usually no more than 40 wt-%, typically no more than 35 wt-%, and often no more than 30 wt-% based on the total weight of the composition. A preferred range for the amount of film former in the coatable composition is 10 to 25 wt-% based on the total weight of the composition.

Typically, the same amounts of film former are present in the film. The film former is present in the film at a level of at least 2 wt-%, and typically at least 5 wt-% by total film weight. Usually, no more than 50 wt-% of the film is the film former, typically no more than 35 wt-% by total film weight. A preferred range for the amount of film former in the film is 10 to 25 wt-% by total film weight.

The preferred film former for an edible film product is hydroxypropyl cellulose, at a level of 15 wt-% of the coatable composition and 15 wt-% of the final film product.

When the amounts of components of the film are discussed herein, it is understood that the amounts of various components of the film can be ascertained in at least two ways. First, the components of the coatable composition that is coated to produce the film can be examined. Typically, the coated film and the film product will have the same components in the same amounts as the coatable composition. Second, the film itself can be chemically analyzed to determine its components.

In one embodiment, neither the edible film product nor the coatable composition includes more than 5 wt-% of other film formers, such as pullulan, starch, carboxymethyl cellulose, pectin or Acacia Senegal. In one embodiment, neither the edible film product nor the coatable composition includes more than 0.5 wt-% of these other film formers. In one embodiment, neither the edible film product nor the coatable composition includes any of these other film formers.

Viscosity Modifier

The coatable composition of the present invention includes a viscosity modifier, lowering the viscosity of the composition, such as a fatty alcohol. The film former is preferably soluble in the fatty alcohol. The fatty alcohol can also be referred to as a viscosity modifier or liquifier. Fatty alcohols are generally water soluble, primary C8-C20 alcohols, usually straight chain. The alcohol can be a saturated or unsaturated alcohol. In one embodiment, the fatty alcohol is an unsaturated alcohol. The fatty alcohol is preferably a thermoplastic material. The fatty alcohol used can be either a solid or liquid at room temperature, however, those solid at room temperature are preferred in order to reduce the cooling and solidifying period of the coatable composition after coating. A sharp melting point and/or crystallization point is desired for the fatty alcohol. Preferably, the melting point is at least 20° C. and no more than 120° C.

Examples of suitable fatty alcohols include stearyl alcohol (also known as 1-octadecanol) which has a melting point of about 57-66° C., myristyl alcohol (also known as 1-tetradecanol) which has a melting point of about 38° C., and cetyl alcohol (also known as 1-hexadecanol) which has a melting point of about 52-54° C., although other fatty alcohols are also be suitable. Mixtures of these fatty alcohols can be used.

The fatty alcohol is included at a percentage weight level of the total film sufficient to cause the film to change from a solid to a liquid within a relatively narrow temperature range of 20° C. or less. The melting point of the fatty alcohol component influences the melting properties of the final film product. Where the fatty alcohol has a fairly sharp melting point, the film will more quickly disperse when in contact with body fluids such as saliva and the film will more quickly solidify and become non-tacky as it cools after extrusion. In addition, the fatty alcohol serves to lower the viscosity of a composition including a film former that is a high molecular weight polymer such as hydroxypropyl cellulose, thereby allowing the coatable composition to be more easily extruded.

Fatty alcohols such as stearyl alcohol cause the film to have a coating effect in the mouth, which is an advantage in some applications. For example, when the film is used for breath freshening, the coating action will cause the active ingredients to be well distributed in the mouth. When the film is used to deliver an analgesic or other medicament for a sore throat, the coating action will help evenly apply the medicament.

The fatty alcohol is present in the coatable composition at a level of at least 10 wt-%, and typically at least 15 wt-% based on the total weight of the composition. Usually, no more than 80 wt-% of the coatable composition is fatty alcohol, typically no more than 60 wt-% based on the total weight of the composition. A preferred range for the amount of fatty alcohol in the coatable composition is 20 to 50 wt-% based on the total weight of the composition. The fatty alcohol is present, as a ratio to the film former, at a level of 1:2 to 3:1 by weight in the coatable composition. Preferably, the ratio of fatty alcohol to film former is 2:1 by weight.

Similar levels of fatty alcohol are present in the film. The fatty alcohol is present in the film at a level of at least 10 wt-%, and typically at least 15 wt-%. Usually, no more than 80 wt-% of the film is the fatty alcohol, typically no more than 60 wt-%. A preferred range for the fatty alcohol in the film is 20 to 50 wt-%. The fatty alcohol is present, as a ratio to the film former, at a level of 1:2 to 3:1 by weight in the final film. Preferably, the ratio of fatty alcohol to film former is 2:1 by weight.

A preferred alcohol for use with hydroxypropyl cellulose is stearyl alcohol. A preferred ratio of stearyl alcohol to hydroxypropyl cellulose is approximately 2:1 by weight.

Fillers or Bulking Agents

The coatable composition of the present invention generally includes a filler or bulking agent to add solids and/or volume to the coatable composition and to the resulting film product. The filler or bulking agent provides a desired “mouth feel” to the film. Preferably, the filler or bulking agent selected dissolves or is soluble in the fatty alcohol and film former.

A carbohydrate is suitable as a filler. The filler can include a carbohydrate, such as a carbohydrate sugar having a molecular weight of at least 100 and at most 2000. Examples of suitable fillers or bulking agents include dextrose, sorbitol, polydextrose, trehalose, maltose, maltodextrin, corn syrup solids, mannitol, xylitol, cyclodextrins, calcium carbonate and mixtures thereof. It is understood that for edible films of the invention, other food grade fillers or bulking agents could be used. For films intended for topical application, it is not necessary that the fillers or bulking agents are food grade.

The bulking agent is preferably mostly tasteless or has a sweet taste and preferably does not have a strong bad taste. The bulking agent can also serve a role as a sweetener, as is the case with dextrose, sorbitol, mannitol, xylitol, cyclodextrins and mixtures thereof. By taking up space in the composition, the bulking agent overcomes the somewhat slimy mouth feel of a viscous film former such as hydroxypropyl cellulose. Preferably the bulking agent does not react with any of the other ingredients of the coatable composition.

The bulking agent will likely have a lower molecular weight than the film former. The molecular weight of dextrose (glucose) is about 180, which includes one glucose ring. The disaccharides, like maltose, lactose, and sucrose, include two glucose rings and have a molecular weight about twice that of dextrose, at about 342. For polydextrose substances with multiple glucose rings the molecular weight could be up to 1000 or 2000. In one embodiment, the bulking agent has a molecular weight of at least 150 and not more than 2000. In another embodiment, the bulking agent has a molecular weight of at least 100 and not more than 600.

In one embodiment, dextrose monohydrate is included in the coatable composition as a bulking agent. Dextrose monohydrate typically includes about 8 wt-% water by weight. Dextrose monohydrate melts at about 185° F. (85° C.) and will not degrade or char until it reaches a temperature of about 265° F. (129° C.) or higher. Anhydrous dextrose can also be used as a bulking agent. In one embodiment, the bulking agent is a material that does not degrade or char when it melts, and preferably, does not degrade or char at a temperature within 10° C. of its melting point. Preferably, the bulking agent does not degrade or char at temperatures of 275° F. (135° C.) or lower. In another embodiment, the bulking agent is a material that does not degrade or char at temperatures of 265° F. (129° C.) or lower.

U.S. Pat. No. 6,596,298 describes an oral care film including a water soluble film-forming polymer and essential oils and emphasizes that oral care films having a high oil content should avoid substantial amounts of humectant in the film, and preferably have no humectant, to avoid producing an overly moist, self-adhering film. However, the coated film of the present invention does not become overly moist or self-adhering when a humectant is included, even when essential oils are included. In one embodiment, the coated film includes up to 40-50 wt-% of dextrose, which is a humectant, without adverse effects. The mixture of hydroxypropyl cellulose, fatty alcohol and dextrose result in a film with desirable characteristics, that is not self-adhering, even when oils are included.

The bulking agent is present in the coatable composition at a level of at least 20 wt-%, and typically at least 25 wt-% based on the total weight of the composition. Usually, no more than 75 wt-% of the coatable composition is the filler or bulking agent, typically no more than 70 wt-%. A preferred range for the amount of filler or bulking agent in the coatable composition is 40 to 60 wt-%.

Typically, the same levels of bulking agent are present in the film as are present in the coatable composition. The bulking agent is present in the film at a level of at least 20 wt-%, and typically at least 25 wt-% by total film weight. Usually, no more than 75 wt-% of the film is the filler or bulking agent. A preferred range for the amount of filler or bulking agent in the film is 40 to 60 wt-% by total film weight.

Sweetener

If the film product is to be orally administered, it is preferred that the coating composition, and the resulting film product, includes a sweetener. Also, if the film product includes a medicament with bitter or undesirable taste, the sweetener can have a masking effect and hide the taste of the medicament. The sweetener can be a natural sweetener or an artificial sweetener or include both.

Examples of natural sweeteners include monosaccharides such as glucose (dextrose), fructose (levulose), galactose, dextrose derivatives, dextrin, dextrin derivatives such as maltodextrin, disaccharides such as sucrose, maltose, lactose and cellobiose, and mixtures of these.

Additional examples of natural sweeteners include polysaccharides such as maltodextrin, starch, amylase and amyl pectin. Further examples of natural sweeteners include xylose, ribose, mannose, invert sugar (a mixture of fructose and glucose derived from sucrose), partially hydrolyzed starch, corn syrup solids, dihydrochalcones, monellin, steviosides, and glycyrrhizin. Crystalline hydrate forming sugars, which include maltose, lactose, and also isomalt, trehalose, and raffinose, can also be suitable natural sweeteners to use. Mixtures of these sweeteners can also be used.

Many different natural and artificial sweeteners can be used. For example, U.S. Pat. No. 6,596,298 describes an oral care film and discusses many different sweeteners that can be included in a film. The film of the present invention can include any of the sweeteners that are described and listed in U.S. Pat. No. 6,596,298. The text of U.S. Pat. No. 6,596,298 is hereby incorporated herein in its entirety.

It is realized that various ingredients can act as both a sweetener and as a filler or bulking agent, ingredients such as dextrose, sorbitol, mannitol, xylitol, cyclodextrins and mixtures of these.

Since a typical breath freshener strip, as illustrated in FIGS. 1 and 2 weighs only approximately 0.3 grams, it is difficult to incorporate a sufficient amount of natural sweetener to give the desired “burst” of sweetness. Thus preferably, the sweetener used for the coating composition includes an artificial sweetener, which are significantly more sweet than sugar. For example, saccharin is about 300 times sweeter than sugar, acesulfame K is about 200 times as sweet as sugar, sucralose has about 450 to 650 times the sweetness of sugar, and aspartame is about 200 times as sweet as sugar.

The sweetener is present in the coatable composition at a level of at least 0.5 wt-%, and typically at least 1 wt-% based on the total weight of the composition. Usually, no more than 25 wt-% of the coatable composition is the sweetener, typically no more than 20 wt-% based on the total weight of the composition. A preferred range for the amount of sweetener in the coatable composition is 2 to 10 wt-% based on the total weight of the composition.

Typically, similar levels of sweetener are present in the coated film. The sweetener is present in the film at a level of at least 0.5 wt-%, and typically at least 1 wt-% by total film weight. Usually, no more than 25 wt-% of the film is the sweetener. A preferred range for the amount of sweetener in the film is 2 to 10 wt-% by total film weight.

Combined Sweetener and Bulking Agent

To facilitate processing, the sweetener and bulking agent can be obtained combined, and used in such form. Examples of suitable materials which are a combination of a sweetener and a bulking agent or filler include SWEET ONE® sugar substitute from Stadt Corp., which includes approximately 85-98 wt-% dextrose and 2-15 wt-% acesulfame K, and SPLENDA® sweetener from McNeil Specialty Products Company, which is sucralose with a maltodextrin carrier. Mixtures of these materials can also be used. Although the carriers, such as dextrose and maltodextrin, can be a sweetener in their own right, it is understood that the sweetening effect from products such as SWEET ONE® sugar substitute and SPLENDA® sweetener results mainly from the artificial sweetener component.

The combination of sweetener/bulking agent is present in the coatable composition at a level of at least 20 wt-%, and typically at least 35 wt-% based on the total weight of the composition. Usually, no more than 75 wt-% of the coatable composition is the sweetener/bulking agent, typically no more than 60 wt-% based on the total weight of the composition. A preferred range for the sweetener/bulking agent in the coatable composition is 45 to 55 wt-% based on the total weight of the composition.

Similarly, the sweetener/bulking agent is present in the film at a level of at least 20 wt-%, and typically at least 35 wt-% by total film weight. Usually, no more than 75 wt-% of the film is sweetener/bulking agent and typically no more than 60 wt-% by total film weight. A preferred range for the sweetener/bulking agent in the film is 45 to 55 wt-% by total film weight.

Adjuvants

The film product will typically include other ingredients selected based on the resulting use of the film product. For example, edible films can include a flavorant, such as peppermint, menthol, or other suitable flavor for the film. Medicinal films, whether edible or for topical application, will include a medicament. Nutrients such as vitamins and minerals can be included in edible films

Adjuvants such as flavorants, medicaments, nutrients and other additives such as color (dyes, pigments), can be added to the coatable composition so that the resulting film includes the additive. Many flavorants, medicaments, nutrients and dyes are water based products. It is preferred that any water-based additives are used in levels to not appreciably disrupt the thermoplastic properties of the coatable composition.

By use of the term “flavorant”, what is intended is an ingredient that imparts a desired flavor and/or odor to the film product. Examples of usable flavorants include natural oils and essential oils. For example, peppermint oil, menthol, eucalyptol, cinnamon flavoring, clove oil and other oils, vanillin, cacao, fruit extracts, mixtures thereof, and the like can be used. Artificial flavors can also be used.

It is understood that generally flavorant can be used. For example, U.S. Pat. No. 6,596,298 describes an oral care film and discusses many different flavorants that can be included in a film. The film of the present invention can include any of the flavorants that are described and listed in U.S. Pat. No. 6,596,298, which was previously incorporated herein.

Another type of adjuvant that can be present in a film intended for oral use is a masking agent, designed to mask an undesirable taste. A sweetener can be used to mask other flavors, or other masking agents can be included.

Essential oils are generally compatible with the film former hydroxypropyl cellulose and fatty alcohol, so that a coatable composition including these three elements can be mixed together easily. As a result, it is typically not necessary to utilize an emulsification process to combine the essential oil, component hydroxypropyl cellulose and fatty alcohol.

The amount of adjuvants, in the coatable composition is typically no more than 20 wt-% of the coatable composition, typically no more than 10 wt-% by total weight of the composition. A preferred range for the amount of such additives in the coatable composition is 0.5 to 5 wt-%, more preferred 1 to 2 wt-% by total weight of the composition. However, for some applications a medicament is as much as 40 wt-% by the total weight of the coatable composition.

Similar amounts of these ingredients are found in the film. The amount of flavorants, colorants, medicaments, if present, in the final film product is no more than 20 wt-%, typically no more than 10 wt-% by total film weight. A preferred range is 0.5 to 5 wt-%, more preferred 1 to 2 wt-% by total film weight. For some applications, a medicament is as much as 40 wt-% of the film by total film weight.

Alternately, any adjuvants can be coated onto the film product after production of the film. An additive can be sprayed onto the film after it is coated, either before the film has completely cooled or after it has cooled to the ambient temperature. A coating on the film product can be useful to mask the taste of the active ingredient or to prevent the active ingredient from numbing the tongue or other surfaces in the oral cavity. The coatings that can be used for this purpose are known to those skilled in the art, including polymers, celluloses, such as ethylcellulose, mixtures thereof and the like.

It can be desirable to print something on the surface of the film for many applications. Printing techniques for printing on flexible films are known in the art and could be applied to this film. For films intended for oral consumption, a food-grade ink will be used.

By use of the term “medicament” and variations thereof, what is intended is an ingredient that provides a therapeutic effect. An example of a medicament, for sore throat relief, is benzocaine. For topical applications, aloe or other skin care agents can be included.

Examples of medicaments that can be included in the film include:
A. antimicrobial agents, such as triclosan, cetyl pyridium chloride, domiphen bromide, quaternary ammonium salts, zinc compounds, sanguinarine, fluorides, alexidine, octonidine, EDTA, mixtures thereof and the like,
B. non-steroidal anti-inflammatory drugs, such as aspirin, acetaminophen, ibuprofen, ketoprofen, diflunisal, fenoprofen calcium, naproxen, tolmetin sodium, indomethacin, mixtures thereof and the like,
C. anti-tussives, such as benzonatate, caramiphen edisylate, menthol, dextromethorphan hydrobromide, chlophedianol hydrochloride, mixtures thereof and the like,
D. decongestants, such as pseudoephedrine hydrochloride, phenylepherine, phenylpropanolamine, pseudoephedrine sulfate, mixtures thereof and the like,
E. anti-histamines, such as brompheniramine maleate, chlorpheniramine maleate, carbinoxamine maleate, clemastine fumarate, dexchlorpheniramine maleate, diphenhydramine hydrochloride, diphenylpyraline hydrochloride, azatadine meleate, diphenhydramine citrate, doxylamine succinate, promethazine hydrochloride, pyrilamine maleate, tripelennamine citrate, triprolidine hydrochloride, acrivastine, loratadine, brompheniramine, dexbrompheniramine, mixtures thereof and the like,
F. expectorants, such as guaifenesin, ipecac, potassium iodide, terpin hydrate, mixtures thereof and the like.

Many different medicaments can be included in the film. U.S. Pat. No. 6,596,298 lists many different examples of pharmaceutical agents that can be included in a film. The film of the present invention can include any of the pharmaceutical agents, dose amounts and other substances that are described and listed in U.S. Pat. No. 6,596,298, which was previously incorporated herein.

Preferred Compositions

One preferred coatable composition has the following components where amounts are specified based on the total weight of the composition: 30 wt-% fatty alcohol (i.e., stearyl alcohol), 53.2 wt-% sweetener and filler (i.e., SWEET ONE® sugar substitute) which provides about 4 wt-% acesulfame K and about 49.2 wt-% dextrose, 15 wt-% film former (i.e., hydroxypropyl cellulose), and 1.8 wt-% flavorant (i.e., 0.8 wt-% peppermint oil and 1.0 wt-% menthol). There is no water added to this composition.

Another preferred coatable composition has the following components where amounts are specified based on the total weight of the composition: 35 wt-% fatty alcohol (e.g., stearyl alcohol), 49 wt-% sweetener and filler (i.e., SWEET ONE® sugar substitute) which provides about 3.7 wt-% acesulfame K and about 45.3 wt-% dextrose, 15 wt-% film former (e.g., hydroxypropyl cellulose), and 1 wt-% flavorant (e.g., peppermint oil). No water is added to this composition. Any water present in the composition is from the 6-8% moisture content of the hydroxypropyl cellulose and dextrose. This water is partially evaporated during the mixing steps. In one embodiment, this water is removed from the coatable composition by applying a vacuum during the mixing steps.

The low water content of the film is advantageous. Preferably the water content of the film is 10 wt-% or less, and can be 8 wt-% or less or preferably 6 wt-% or less by total film weight. The water content of the film can be 5 wt-% or less, 2 wt-% or less, 0.5 wt-% or less by total film weight, or the film can lack any detectable water content.

Mixing the Coatable Composition

The coating composition is prepared by combining the desired ingredients in any suitable manner to form a mixture. It is preferred that the resulting coatable composition is thoroughly mixed with a smooth consistency. The ingredients in the coatable composition can be completely solvated, or, the ingredient can be mixed together retaining their individual states. The coatable composition can be homogeneous, although this is not necessary and the coatable composition is not homogenous in one embodiment.

A preferred method for preparing the coatable composition is to mix the film former into the fatty alcohol, which is in a liquid form. If solid, the fatty alcohol is melted or otherwise liquefied prior to adding the film former. The fatty alcohol provides a solvent for dispersing or melting the film former therein. As stated above, preferably the film former dissolves or at least is soluble in the fatty alcohol.

Optional ingredients, such as sweetener and/or bulking agent, can be added to the fatty alcohol either before or after the film former is added. The particular order of addition will depend on the amount and types of ingredients used. In the preferred method, water is not added to the coatable composition during mixing in any appreciable quantity. Although water can be a part of some of the ingredients, such as hydroxypropyl cellulose and dextrose, it is not introduced by itself in quantities of greater than 2 wt-%, and preferably water alone is not introduced at all.

After all ingredients are mixed and prior to extrusion, the coatable composition preferably has a viscosity of less than 150,000 centipoise at 121° C., more preferably 125,000 centipoise.

Extruding the Coatable Composition

The coating composition, as a liquid, is coated to provide a coated film that can be cut into the resulting film product. Examples of suitable coating methods include extrusion coating, knife coating, roll coating, and the like, however due to the thermoplastic nature of the coatable composition, the preferred coating method is by extrusion at elevated temperatures.

Typically, the coatable composition is extruded through a slot die to form the film product. The coatable composition can be provided to the extruder as a liquid and then extruded, or, the coatable composition can be provided to the extruder as a solid material, which is then melted by the extruder or other equipment upstream of the extruder.

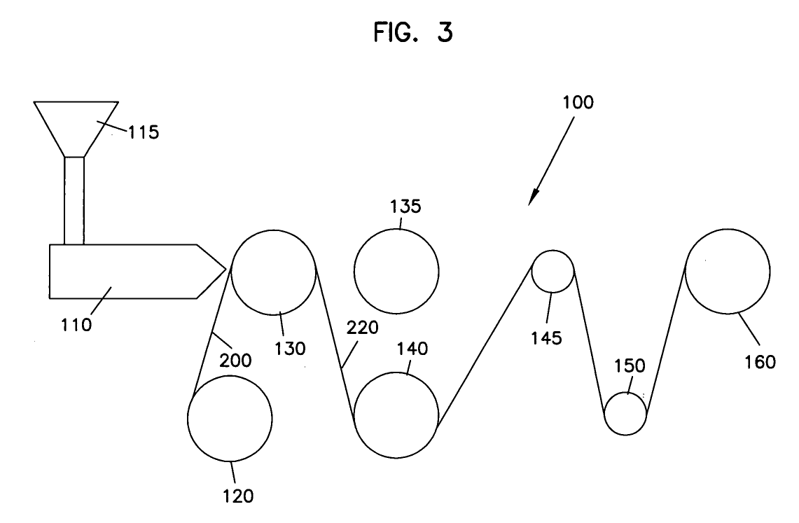

FIG. 3 is a schematic depiction of an exemplary process for coating the coatable composition of the present invention. Coating system 100 is a typical extrusion coating system; those skilled in the art of coating will understand that numerous variations of system 100 could be used for providing the film product of the present invention.

Referring to FIG. 3, system 100 includes an extruder 110 for providing liquid coatable composition. In the system illustrated as 100, the coatable composition is fed to extruder 110 via hopper 115, which retains and feeds solid particulate of the coatable composition to extruder 110. Extruder 110 melts the solid coatable composition to form a coatable liquid. A preferred extruder 110 for such a system is a single screw extruder.

In an alternate coating system, the coatable composition could be fed to extruder 110 in a liquid state. For such a set-up, it can be desirable to feed the coatable composition to extruder 110 via heated pipes or tubes, to inhibit the composition from setting up prior to reaching extruder 110. In yet another alternate coating system, the ingredients for the coatable composition, such as the film former and the fatty alcohol, could be fed individually to extruder 110. Extruder 110 would compound and melt the ingredients; a preferred extruder for such a system would be a twin screw extruder.

The preferred coating method for the thermoplastic coatable composition of the present invention is with a slot die. Slot dies are well known in the art of coating, and include a manifold for receiving liquid material and a slot for expelling the material. Extruder 110 provides a steady supply of liquid material to the manifold to provide a consistent coating through the slot by applying pressure greater than atmospheric temperatures to the liquid material. The liquid material is at a temperature above the melting point of the coating composition; typically, the liquid material is at least 150° F. (66° C.). For a composition including stearyl alcohol, an extrusion temperature of at least 200° F. (93° C.), more preferably at least 225° F. (107° C.) is desired. The liquid material is preferably not heated above 250° F. (122° C.). To provide a final film thickness of 2 mils, the height of the slot is 2 mils or 0.05 mm. Similarly, to provide a film 5 mils or 0.1 mm thick, the height of the slot is 5 mils or 0.1 mm.

Referring again to FIG. 3, system 100 includes a roll 120 of carrier web 200. The coatable composition is extruded onto carrier web 200 by extruder 110. Typically, a back-up roll or contact roll 130 is used; often, contact roll 130 is a driven roll. Coated web 220, which has a coating of coatable composition on carrier web 200, is fed to idler roll 140. A nip roll 135 can be included to facilitate movement of web 220. Nip roll 135 can be any suitable roll, for example, a steel roll or a rubber roll. Roll 135 can be chilled to speed the cooling and solidification of the coatable composition. However, with some coatable compositions, the cooling of the extruded material due to the drop from the extrusion temperature (e.g., 225-250° F., 107-121° C.) to ambient temperature, the extruded film will already have cooled and solidified prior to reaching roll 135.

After extrusion at an elevated temperature, the coatable composition quickly cools to a non-tacky state. It is not necessary to dry the film to drive off moisture or solvents. It is not necessary to apply heat to the film and circulate air for the extruded film to become non-tacky. By “non-tacky”, it is meant that the film does not have an appreciable tack or stickiness upon touching it at typical ambient temperatures of 21-23° C. The non-tacky film does not significantly deform and adhere to other surfaces at temperatures of 21-23° C. Preferably the film is also non-blocking, so that it does not stick to itself. As a result, the film can be wound without a top liner in one embodiment. Because the film becomes non-tacky simply by cooling, and drying steps are not necessary, the film can be extruded at faster line speeds compared to films that require drying or driving off solvents. Also, the coated film can be easily separated from the carrier web substrate.

System 100 includes various other rolls, such as rolls 145, 150. Such rolls are known to control the tension of web 220. One skilled in the art of coating will be able to design an appropriate set up. Web 220 is collected as roll 160.

The resulting coated film product has a thickness of 1 mil to 5 mil (0.03 to 0.1 mm). For an edible film product, a thickness of 2 mil (0.05 mm) is preferred.

Run speeds for system 100 are generally at least 10 feet per minute and typically at least 50 feet per minute. Speeds of 100 feet per minute and more would be typical for commercial coating processes. The nature of the coatable composition, being a thermoplastic, allows the extruded film to solidify within 10 seconds of coating. Depending on the specific composition, solidification can occur within 5 seconds of coating, and even within 1 second of coating. Coating compositions having lower amounts of water, e.g., no more than 5 wt-% by total composition weight, will dry or solidify quicker than compositions having higher amounts of water. Once solidified, the film product is non-tacky to the touch. As the film further crystallizes, the film becomes more rigid and handleable.

The resulting web of film can be slit using conventional techniques. Due to the thermoplastic properties of the film, excess material, such as the head and tail of the web and the side edges, can be removed from the usable film and recycled to the extruder. That is, the film can be remelted and coated again.

### EXAMPLES

The following examples are not limiting, but are provided for a better understanding of the invention. All percentages and parts are provided as weights, based on the weight of the coatable composition unless otherwise noted.

### Example 1

First, 600 grams of stearyl alcohol (commercially available from Croda under the designation “Crodacol S-95 NF”) was melted in a Sigma high shear mixer at a temperature of 225° F. To this melted fatty alcohol were added 500 grams of a mixture of dextrose and acesulfame K (commercially available from Stadt Corp. under the designation “Sweet One”). The mixture was stirred and heated at 200-225° F. until the dextrose melted. Acesulfame was present dispersed in the melted mixture of dextrose and fatty alcohol. To this, 300 grams of hydroxypropyl cellulose (commercially available from Hercules under the designation KLUCEL® EF polymer) were added and mixed at 225° F. until thickened. An additional 564 grams of SWEET ONE® sugar substitute were added and mixed at about 225° F. The mixture appeared a little grainy. Next, 16 grams of peppermint oil and 20 grams of menthol were added to the mix; the resulting mix was smoother with the oils added. The coatable composition was fairly thick, with a viscosity of about 125,000 centipoise at 121° C.

The coatable composition had 30 wt-% fatty alcohol (i.e., stearyl alcohol), 4 wt-% sweetener (i.e., acesulfame K), 49.2 wt-% filler (i.e., dextrose), 15 wt-% film former (i.e., hydroxypropyl cellulose), and 1.8 wt-% flavorant based on the total weight of the composition. No water was added to the coatable composition.

The coatable composition was pulled with a metal ring to a thickness of 0.05 mm (2 mil) onto silicone release paper. Within 1 second of being coated, the film composition had hardened and was non-tacky to the touch. After 10 seconds, the film was cut to about 2.5 cm by 5.1 cm (1 inch by 2 inch) and removed from the wax paper. The film was sufficiently rigid and self-supporting to withstand handling.

The film was tasted and had a peppermint flavor. The film dissolved on the tongue within 2 seconds and left no undesirable aftertaste or sticky or pasty feeling.

### Example 2

Example 2 was prepared similar to Example 1, except that 564 grams of the dextrose/acesulfame K were added to the melted stearyl alcohol rather than 500 grams. This mixture required longer heated mixing than Example 1. The remaining 500 grams of the dextrose/acesulfame K were added after the 300 grams of hydroxypropyl cellulose.

The coatable composition had 30 wt-% fatty alcohol (i.e., stearyl alcohol), 4 wt-% sweetener (i.e., acesulfame K), 49.2 wt-% filler (i.e., dextrose), 15 wt-% film former (i.e., hydroxypropyl cellulose), and 1.8 wt-% flavorant based on the total weight of the coatable composition.

Although the resulting coatable composition looked the same as that from Example 1, this coatable composition was not coated to form a film.

### Example 3

First, 700 grams of stearyl alcohol were melted. To this melted fatty alcohol were added 980 grams of SWEET ONE® sugar substitute. To this, 300 grams of hydroxypropyl cellulose were added. Next, about 2 grams of peppermint oil were added to the mix.

The coatable composition had 35 wt-% fatty alcohol (i.e., stearyl alcohol), 3.7 wt-% sweetener (i.e., acesulfame K), 45.3 wt-% filler (i.e., dextrose), 15 wt-% film former (i.e., hydroxypropyl cellulose), and 1 wt-% flavorant, where the quantities are based on the total weight of the composition. No water was added to this composition.

The coatable composition was coated as described in Example 1.

Various modifications and alterations of this invention will become apparent to those skilled in the art without departing from the scope and principles of this invention, and it should be understood that this invention is not to be unduly limited to the illustrative embodiments set forth hereinabove.

It is to be understood, however, that even though numerous characteristics and advantages of the present disclosure have been set forth in the foregoing description, together with details of the coatable compositions and the film products, the disclosure is illustrative only, and changes can be made in detail, especially in matters of shape, size and arrangement of parts and types of materials within the principles of the disclosure to the full extent indicated by the broad general meaning of the terms in which the appended claims are expressed.

1. A film resulting from a coating of a composition comprising:
(a) hydroxypropyl cellulose; and
(b) at least 10 wt-% fatty alcohol component by total film weight;
(c) wherein the film is thermoplastic;
(d) wherein the film is configured to disperse in contact with a body fluid of a live human or other animal in 5 minutes or less;
(e) wherein the film has a thickness of 1 mm or less;
(f) wherein the film has a perimeter area of 15 square cm or less.

2. The film according to claim 1:
(a) wherein the fatty alcohol component has a melting point at a temperature at least 20° C. and at most 120° C.

3. The film according to claim 1:
(a) wherein the fatty alcohol component is included at a percentage weight level of the total film sufficient to cause the film to change from a solid to a liquid within a temperature range of 20° C or less.

4. The film according to claim 1:
(a) wherein the fatty alcohol component includes a straight-chain C8 to C20 alcohol.

5. The film according to claim 4:
(a) wherein the fatty alcohol component includes at least one alcohol selected from the group consisting of myristyl alcohol, cetyl alcohol, stearyl alcohol, and mixtures thereof.

6. The film according to claim 1:
(a) wherein the fatty alcohol component includes stearyl alcohol.

7. The film according to claim 1, the composition further comprising:
(a) flavorant.

8. The film according to claim 7:
(a) wherein the flavorant includes essential oil component.

9. The film according to claim 8:
(a) wherein the essential oil component includes at least one selected from the group consisting of menthol, peppermint oil, eucalyptol, cinnamon oil and mixtures thereof.

10. The film according to claim 1, the composition further comprising:
(a) bulking agent.

11. The film according to claim 10:
(a) wherein the bulking agent includes carbohydrate.

12. The film according to claim 11:
(a) wherein the bulking agent includes carbohydrate sugar having a molecular weight of at least 150 and not more than 600.

13. The film according to claim 10:
(a) wherein the bulking agent includes dextrose.

14. The film according to claim 1, the composition further comprising:
(a) sweetener.

15. The film according to claim 1, the composition further comprising:
(a) medicament.

16. The film according to claim 1:
(a) wherein the film is rectangular and has a width of 3 cm or less and a length of 4 cm or less.

17. The film according to claim 1:
(a) wherein a perimeter area of the film is 10 square centimeters or less.

18. The film according to claim 1 wherein the perimeter area is at least 4 square centimeters.

19. The film according to claim 1:
(a) wherein the film is orally ingestible and
(b) wherein the film is configured to disperse in a mouth of a consumer within 30 seconds or less.

20. The film according to claim 1 wherein:
(a) the film thickness is at least 0.03 mm and at most 0.1 mm.

21. The film according to claim 1 the composition comprising:
(a) 5 to 50 wt-% hydroxypropyl cellulose by total film weight; and
(b) 15 to 60 wt-% fatty alcohol by total film weight.

22. The film according to claim 1 the composition comprising:
(a) 10 to 25 wt-% hydroxypropyl cellulose by total film weight;
(b) 20 to 50 wt-% stearyl alcohol by total film weight;
(c) 40 to 60 wt-% dextrose by total film weight; and
(d) 1 to 10 wt-% artificial sweetener by total film weight.

23. The film according to claim 22 the composition further comprising:
(a) 0.5 to 5 wt-% flavorant by total film weight.

24. The film according to claim 23 wherein:
(a) the flavorant includes essential oil component.

25. A film resulting from a coating of a composition comprising:
(a) 5 to 50 wt-% hydroxypropyl cellulose by total film weight;
(b) 5 to 60 wt-% fatty alcohol component by total film weight;
(c) 15 to 80 wt-% dextrose by total film weight; and
(d) wherein the film is thermoplastic;
(e) wherein the film has a thickness of 1 mm or less; and
(f) wherein the film has a perimeter area of 15 square cm or less.

26. The film according to claim 25:
(a) wherein the film thickness is at least 0.03 mm and at most 0.1 mm.

27. The film according to claim 26:
(a) wherein the fatty alcohol component has a melting point at a temperature at least 20 degrees C. and at most 120 degrees C.

28. The film according to claim 27:
(a) wherein the fatty alcohol component is included at a percentage weight level of the total film sufficient to cause the film to change from a solid to a liquid within a temperature range of 20 degrees C.

29. The film according to claim 28:
(a) wherein the fatty alcohol component includes a straight-chain C8-C20 alcohol.

30. The film according to claim 29:
(a) wherein the fatty alcohol component includes at least one selected from the group consisting of myristyl alcohol, cetyl alcohol, stearyl alcohol and mixtures thereof.

31. The film according to claim 30 the composition further comprising:
(a) 1 to 20 wt-% artificial sweetener by total film weight;
(b) wherein the film is orally ingestible and
(c) wherein the film is configured to disperse in a mouth of a consumer within 30 seconds or less.

32. The film according to claim 31 wherein:
(a) the film has a thickness of 0.03 to 0.1 mm.

33. A film resulting from a coating of a composition comprising:
(a) 10 to 25 wt-% hydroxypropyl cellulose by total film weight;
(b) 20 to 50 wt-% stearyl alcohol by total film weight;
(c) 40 to 60 wt-% dextrose by total film weight; and
(d) 1 to 5 wt-% artificial sweetener by total film weight;
(e) wherein the film has a thickness of 0.03 to 0.1 mm.
(f) wherein the film is thermoplastic;
(g) wherein the film is orally ingestible; and
(h) wherein the film is configured to disperse in a mouth of a consumer within 30 seconds or less; and
(i) wherein a perimeter area of the film is 15 square centimeters or less.

34. A method of making a film, the method including the steps of:
(a) providing a water-soluble film former;
(b) mixing the film former in a fatty alcohol component to form a mixture; and
(c) extruding the mixture to form a coated film, wherein the mixture is at a temperature of at least 65 degrees C. when extruded.

35. The method according to claim 34:
(a) wherein the fatty alcohol component melts at a temperature of at least 20 degrees C. and less than 120 degrees C.

36. The method according to claim 34:
(a) wherein the fatty alcohol component is included at a percentage weight level of the total film sufficient to cause the mixture to have a viscosity of 150,000 centipoise or less.

37. The method according to claim 34:
(a) wherein the fatty alcohol component includes a straight-chain C8-C20 alcohol.

38. The method according to claim 34:
(a) wherein the fatty alcohol component includes at least one selected from the group consisting of myristyl alcohol, cetyl alcohol, stearyl alcohol and mixtures thereof.

39. The method according to claim 34, wherein the step of mixing the film former in a fatty alcohol comprises:
(a) mixing the film former in liquid stearyl alcohol.

40. The method according to claim 34, wherein:
(a) the water soluble film former is thermoplastic.

41. The method according to claim 40, wherein the step of providing a water soluble film former comprises:
(a) providing hydroxypropyl cellulose.

42. The method according to claim 34, wherein the step of extruding the mixture comprises:
(a) extruding the mixture through a slot die.

43. The method according to claim 34, further including the steps of:
(a) prior to extruding the mixture to form the coated film, solidifying the mixture to form a solid material;
(b) melting the solid material to form a melted material; and
(c) extruding the melted material.

44. The method according to claim 34, further including the steps of, after extruding the mixture:
(a) trimming away a portion of the coated film at edges of the coated film;
(b) melting the trimmed-away portion of the coated film to form a melted material; and then
(c) extruding the melted material to form a second coated film.

45. A film formed by the method including the steps of:
(a) providing a water-soluble film former;
(b) mixing the film former in a fatty alcohol component to form a mixture, wherein the fatty alcohol component is at least 10 wt-% by total film weight; and
(c) extruding the mixture to form a coated film, wherein the mixture is at a temperature of at least degrees 65 degrees C. when extruded.
(d) when the coated film cools to 30 degrees C. or lower, the extruded film is non-tacky.

46. The film according to claim 45, wherein:
(a) the film former includes liquid stearyl alcohol.

47. The film according to claim 46, wherein:
(a) the water soluble film former is thermoplastic.

48. The film according to claim 47, wherein:
(a) the water soluble film former is hydroxypropyl cellulose.

49. The film according to claim 45, wherein:
(a) the mixture is extruded through a slot die.

50. A method of making a film, the method including the steps of:
(a) providing a water-soluble film former;
(b) mixing the film former with a substance to form a mixture having less than 5 wt-% water; and
(c) extruding the mixture to form a coated film, wherein the mixture is at a temperature of at least 65 degrees C. when extruded.

51. The method according to claim 50, wherein the step of mixing the film former to form the mixture comprises:
(a) mixing the film former with a fatty alcohol.

52. The method according to claim 51, wherein the step of mixing the film former with a fatty alcohol comprises:
(a) mixing the film former with a fatty alcohol component that has a melting point of at least 20° C. and not greater than 120° C.

53. The method according to claim 52, wherein the step of mixing the film former with a fatty alcohol comprises:
(a) mixing the film former with stearyl alcohol.

54. The method according to claim 50, wherein the step of providing a water soluble film former comprises:
(a) providing hydroxypropyl cellulose.

55. The method according to claim 50, wherein the step of extruding the mixture comprises:
(a) extruding the mixture through a slot die.
